(19) United States
(12) Patent Application Publication
(10) Pub. No.: US 2006/0084629 A1
(43) Pub. Date: Apr. 20, 2006

(54) Immune system activating formula composed of selected long chain polysaccharides from natural sources

(76) Inventors: Alvin Needleman, Las Vegas, NV (US); Harriet Needleman, Las Vegas, NV (US)

Correspondence Address: WEIDE & MILLER, LTD., 7251 W. LAKE MEAD BLVD., SUITE 530, LAS VEGAS, NV, 89128, US

(21) Appl. No.: 10/966,175

(22) Filed: Oct. 15, 2004

(51) Int. Cl. A61K 31/715; A61K 36/886

(52) U.S. Cl. 514/54; 424/744

(57) ABSTRACT

A combination of two, specialized, high molecular weight-long chain fractions isolated from Aloe Vera and Maitake TD to stimulate immune system activity comprise long chain-high molecular weight polysaccharides which activate the body's natural immune response, triggering an increase in the production of macrophages, T-cells, B-cells, natural killer cells, cytokines and antibodies. Long chain polysaccharides acquired from different source materials (Aloe Vera, Maitake) generate different expressions of cytokines in turn effecting the production of a larger variety of antibodies against specific and non-specific antigens. These long chain polysaccharides together with other active ingredients may provide proper immune system support thereby preventing debilitating diseases such as cancer, heart disease and aging.

### REFERENCES CITED [REFERENCED BY}

### U.S. Patent Documents

U.S. Pat. No. 6,133,440 October, 2000 Qui, Mahiou

U.S. Pat. No. 6,436,679 August, 2002 Qui, Mahiou

U.S. Pat. No. 6,271,214 August, 2001 Qui, Mahiou

WO 02/03999 January, 2002 Pasco, Pugh, Ross, Elsouhy

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

1. Field of the Invention

This invention relates to a method for developing nutraceutical products consisting of specialized long chain polysaccharides from natural sources. The select polysaccharides collectively contribute to powerful immunosupportive activity.

2. Prior Art

The immune system is the natural physiological defense that protects the body from toxins, viruses, bacteria and other pathogens that enter the physical environment causing harm and imbalance. A healthy, balanced immune system plays a vital role in restoring depleted energy, stamina, general mood and mental coherence. A healthy immune system translates into a positive population impact financially and socially. Less frequent illness translates to less money spent on medication and fewer sick days without compensation. A better functioning physiology (body systems), prevents future health problems e.g., diabetes, heart disease and arthritis. A better functioning Immune System improves intestinal health allowing better absorption and digestion of nutrients and more efficient efforts to manage weight.

How Does the Immune System Work?

A complex question, one that scientists have barely begun to understand and explore. What is known, is that the immune system is involved in virtually every function of the body including physical, emotional, sexual, hormonal and metabolic pathways. The immune system is affected by many factors, including, but not limited to: stress, mood, exercise, illness, environment, diet, pregnancy, bereavement, age, hobbies, laughter, meditation and medication

Because so many factors can affect the immune system, there is a major challenge keeping healthy and balanced. The key to immune health is finding and maintaining equilibrium. A healthy immune system is balanced and aligned, this means (a) proper balance of immune products and cells, (b) the body is protected from foreign pathogens, (c) supports healing and wellness, (d) promotes good mental attitude, (e) healthy absorption of nutrients.

The invention has the proven ability to restore balanced, healthy function to the immune system.

Unbalanced Immune System

When the immune system becomes unbalanced, the body must work harder to re-shift the balance in the proper direction. While the immune system may be concentrated on one incursion, the system may become vulnerable to an invasive process from another source (environmental-biological or chemical). A vicious cycle may ensue rendering the body weaker (compromised). As an example, immune system stress caused by a massive bacterial invasion, as yeast bacteria would inflict, or simply after a trip when the body is compromised by less sleep, rich food, alcohol, low humidity air on an airplane, and the residual bacterial environment in a hotel room, causing the immune system to become over-active as it aggressively attacks the “antigen storm” to protect the body. When an immune system is over-taxed it is not balanced and becomes vulnerable, making it difficult for physiological corrections to take place.

The invention, using two high molecular weight long chain polysaccharides from natural sources, is successful in stimulating a range of diverse cytokine expressions necessary to generate a maximum number of offensive types of antibodies to neutralize a variety of invading antigens.

Under active Immune Systems and the invention: the invention corrects (re-balances) an under active immune system, which does not respond as it was designed to do. The body simply does not have the ability to adequately attack foreign invaders before they can multiply, mutate and cause great harm and weakness. General symptoms of an under active immune system are infections, colds and emotional distress.

### BACKGROUND OF THE INVENTION—OBJECTS AND ADVANTAGES

The invention consists of a proprietary blend of two long chain polysaccharide fractions from naturally occurring sources, Aloe Vera and Maitake. The polysaccharides identified by the invention generate powerful immunosupportive activity. The effectiveness of the active components; tested—validated and proven by the University of Mississippi, National Center for the Development of Natural Products, University, MS 38655 and four (4) U.S. patents relating to the effectiveness of the long chain polysaccharides as robust immune system stimulants. Additional activity by the Japanese on Maitake D fractions at a phase three level for the past three decades showcases the immunostimulatory importance of the long chain polysaccharide from Maitake.

Description

The active formula components are long chain polysaccharides from: Aloe Vera “Aloeride®” with a molecular weight of 2M to 7M Daltons and “Griffon-Pro™” Maitake TD-fraction® with a molecular weight of >1M Daltons.

An “active” inter-cellular transport system consisting of: Pyridoxine (Vitamin B6), Riboflavin (Vitamin B2), Folate (Folic acid), Cyanocobalamin (Vitamin B12) and Zinc (Zinc oxide) to accelerate tissue and cellular absorption of the active polysaccharides.

An “active” absorption system; Ascorbic acid (Vitamin C) to facilitate intestinal absorption and maintain the structural integrity of long chain, high molecular weight, polysaccharides.

### DETAILS AND DESCRIPTIONS OF THE PREFERRED EMBODIMENTS

The active components of the invention are long chain polysaccharides from: Aloe Vera known as “Aloeride®” with a molecular weight of 2M to 7M Daltons and “Griffon-Pro™” Maitake TD-fraction® with a molecular weight of >1M Daltons.

An “active” transport system; Pyridoxine (Vitamin B6), Riboflavin (Vitamin B2), Folate (Folic acid), Cyanocobalamin (Vitamin B12) and Zinc (Zinc oxide) to enhance tissue and cellular absorption of the active polysaccharides.

An “active” absorption system; Ascorbic acid (Vitamin C) to facilitate intestinal absorption and maintain the structural integrity of long chain, high molecular weight, polysaccharides.

In vitro and in vivo studies have demonstrated that the invention has a preferential effect on human immune system genetic cell lines (NF Kappa B) in studies by the University of Mississippi, Patent WO 02/03999 A1 “High Molecular Weight Polysaccharide Fraction From Aloe Vera With Immunostimulatory Activity”.

“Aloeride®” consisting of four major glycosyl carbohydrates, glucose, galactose, mannose and arabinose, is taken orally ranging from 20 mg to 100 mg., in beverages ranging from 5% to 10% and topically in a range from 1% to 3%. Validation for immune system stimulation is accomplished using a transcription factor based bioassay for nuclear factor kappa β (NF-kappa β) activation in THP-1 human monocytes/macrophages. At 0.5 μg/mL, “Aloeride” (Aloe Vera A fraction) robustly increased NF-kappa β directed luciferase expression to levels 50% of those achieved by maximal concentrations (10 μg/mL) of bacterial lipopolysaccharide (LPS)!

Griffon-Pro™ ( Maitake TD-Fraction®); D-fraction is a highly standardized and purified beta-1,6 glucan (1,6 main chain with 1,3 branches in its chemical structure) and beta-1,3 glucan (1,3 main chain with 1,6 branches in its chemical structure) with about 30% of protein, extracted from Maitake mushroom fruit body. D-fraction has the unique polysaccharide compound. The degree of branching is greater than that of 1,3 beta-glucan often found in other medicinal mushrooms. Researchers theorize that the complexity of branching may make Maitake superior to other beta-glucan products. An essential fact about the D-fraction is that it is effective when consumed orally in a range of 50 mg to 200 mg, the department of Urology at the New York Medical College, Valhalla, N.Y. concluded that the 1,6 beta glucan carrying 1,3 branches (a polysaccharide string of glucose molecules), Grifron Pro D-Fraction (GD), extracted from Maitake mushroom, “demonstrated a cytotoxic effect via oxidative stress on prostatic cancer cells in vitro, leading to apoptosis. For the past three decades, the Japanese continue D-factor research at an FDA phase three level intensity. Dr. Cun Zhuang and Dr. Takashi Mizuno in their research publication “Biological Responses From Grifola frondosa” identify the ability of D-factor to maximize host-mediated response. The D-factor polysaccharides have been shown to potentiate innate (non-specific) and acquired (specific) immune responses and activate several types of immune cells that are vital to homeostasis. Stimulated immunosupportive activities include cytotoxic macrophages, monocytes, neutrophils, natural killer cells, dendritic cells, chemical messengers, cytokines (i.e. interleukin and interferon) lymphocytes (governing antibody production (β-cells) and cell-mediated cytotoxicity (T-cells).

Transport components;

Riboflavin, (Vitamin B2) in a range of 1 mg to 10 mg, helps break down carbohydrates, fats and protein, converts carbohydrates into adenosine triphosphate (ATP). Vitamin B2 promotes the absorption or activation of iron, folic acid and Vitamins B1, B3 and B6 and the production of red blood cells and antibodies. Vitamin B2 acts as an integral part of the support mechanism for the transport of polysaccharides (carbohydrates) into the human cell.

Pyridoxine: in a range of 1 mg to 10 mg, one of the vitamin B6 groups (which also include pyridoxal and pyridoxamine) is transformed in the body to pyridoxal phosphate, which functions as a coenzyme, a substance that enhances the action of an enzyme and thereby helps catalyze and speed a biochemical reaction. The vitamin B6 group is especially important to the function of the central nervous system, skin, and blood. Vitamin B6 is involved in the formation of red blood cells since pyridoxal phosphate is the rate-limiting substance in making heme, a component of hemoglobin, the key oxygen-carrying pigment in red blood cells.

Cyanocobalamin (Vitamin B12); in a range of 1 Mcg to 100 mcg, provides for the normal formation of red blood cells and the health of the nerve tissues. An essential component of the polysaccharide transport and absorption system.

Folate (Folic acid) as Vitamin B12 (Cyanocobalamin); in a range of 5 mcg to 100 mcg, one of the B vitamins that are a key factor in the synthesis of nucleic acid (DNA and RNA).

Zinc Oxide; in a range of 2 mg to 20 mg, a constituent of many enzymes that permit chemical reactions to proceed at normal rates. It is involved in the manufacture of protein and in cell division. Zinc is a constituent of insulin and is concerned with the sense of smell.

Absorption component; Ascorbic Acid (Vitamin C) in a range of 10 mg to 150 mg, weakens the —C—C—C— bonds between carbon atoms on long chain polysaccharides, without altering chemical/physiological properties, allowing for rapid absorption and stimulation of the “Immuno-stimulatory receptor patch” located in the anterior portion of the small intestine.

1-27. (canceled)

28. An immunostimulatory composition comprising, in combination: at least two long chain-high molecular weight polysaccharides from Aloe Vera and Maitake TD isolated fractions providing an improvement in conditions resulting from an impaired or compromised immune system which causes at least one of Acne, Cold Sores, Herpes Simplex, Allergies, High Cholesterol, Free Radical Damage, High Blood Pressure, Gastrointestinal distress and poor digestion, several types of cancer, tumors, Bronchitis, proliferation of Yeast infections, Sleep disorders, Prostate inflammatory diseases, Colds and Flu, Bacterial infections, Viral infections, Lost stamina, Obesity, Aging, Breast cancer, Stress, Post chemotherapy, Radiation therapy, HIV, AIDS and interferences with Wound healing.

29. A therapeutically active immunostimulatory composition comprising, in combination:
(a) at least one long chain Aloe Vera polysaccharide extract having a molecular weight of about 2M to about 7M Daltons;
(b) at least one long chain Maitake TD polysaccharide extract having a molecular weight of about 1M Daltons; and
(c) at least one of an intercellular transport system and an intestinal absorption system, said immunostimulatory composition providing immunosupportive activity to a person when used by said person.

30. The composition of claim 29 further comprising at least about 20 mg of said at least one long chain Aloe Vera polysaccharide extract when orally ingested by said person.

31. The composition of claim 29 further comprising about 20 mg to about 100 mg of said at least one long chain Aloe Vera polysaccharide extract when orally ingested by said person.

32. The composition of claim 29 further comprising about 5% to about 10% of said at least one long chain Aloe Vera polysaccharide extract when orally ingested in a beverage by said person.

33. The composition of claim 29 further comprising about 1% to about 3% of said at least one long chain Aloe Vera polysaccharide extract when topically applied to a portion of said person.

34. The composition of claim 29 further comprising at least about 50 mg of said at least one long chain Maitake TD polysaccharide extract when orally ingested by said person.

35. The composition of claim 29 further comprising about 50 mg to about 200 mg of said at least one long chain Maitake TD polysaccharide extract when orally ingested by said person.

36. The composition of claim 29 wherein said at least one long chain Aloe Vera polysaccharide extract is a glycosyl carbohydrate selected from the group consisting of glucose, galactose, mannose and arabinose.

37. The composition of claim 29 wherein said at least one long chain Maitake TD polysaccharide extract comprises about 30% protein extract of Maitake mushroom fruit body and a glucan selected from the group consisting of a main chain of beta-1,6 glucan having 1,3 branches and a main chain of beta-1,3 glucan having beta-1,6 branches.

38. The composition of claim 29 wherein said at least one transport system is selected from the group consisting of Riboflavin, Pyridoxine, Cyanocobalamin, Folate and Zinc Oxide.

39. The composition of claim 38 wherein said at least one inter-cellular transport system accelerates tissue and cellular absorption of at least one of said at least one long chain Aloe Vera extract polysaccharide and said at least one long chain Maitake TD extract polysaccharide.

40. The composition of claim 29 wherein said at least one intestinal absorption system comprises ascorbic acid.

41. The composition of claim 40 wherein said at least one intestinal absorption system facilitates intestinal absorption and maintains the structural integrity of at least one of said at least one long chain Aloe Vera extract polysaccharide and said at least one long chain Maitake TD extract polysaccharide.

42. The composition of claim 29 further comprising about 1 mg to about 10 mg of Riboflavin.

43. The composition of claim 29 further comprising about 1 mg to about 10 mg of Pyridoxine.

44. The composition of claim 29 further comprising about 1 mcg to about 100 mcg of Cyanocobalamin.

45. The composition of claim 29 further comprising about 5 mcg to about 100 mcg of Folate.

46. The composition of claim 29 further comprising about 2 mg to about 20 mg of Zinc Oxide.

47. The composition of claim 29 further comprising about 10 mg to about 150 mg of ascorbic acid.
